(19) United States
(12) Patent Application Publication
(10) Pub. No.: US 2005/0055037 A1
(43) Pub. Date: Mar. 10, 2005

(54) Suture apparatus and method for sternal closure

(76) Inventor: William Frederick Fathauer JR., Scottsdale, AZ (US)

Correspondence Address: WILLIAM F. FATHAUER, JR., 10390 E. JENAN DR., SCOTTSDALE, AZ, 85260, US

(21) Appl. No.: 10/658,476

(22) Filed: Sep. 9, 2003

(51) Int. Cl. A61B 17/04

(52) U.S. Cl. 606/144

(57) ABSTRACT

A needle driving apparatus for sternal closure following sternotomy. Accessory elements include: 1) a straight surgical needle absent attached suture and 2) a separate suture carrying small cylinders at opposing ends. Apparatus provides a dual capability of 1) needle penetration of sternum from top surface and 2) precise positioning of cylinder at undersurface of sternum. The apparatus is a handled framework supporting a rachet type drive assembly which includes a lever, a pawl, biasing springs and a toothed arm which carries a needle. A framework extension carries a socketed fixture. The fixture precisely positions a cylinder and its suture in the socket. When the lever is taken up by hand, motion is imparted to the arm and needle. The needle penetrates the sternum, then the cylinder, where it becomes locked therein by friction. Retrograde extraction of needle (using a conventional needle driver) delivers the suture by shuttle action to its desired position, atop the sternum. Repetition on the procedure described, on the opposing side of the sternum completes the placement of a single suture.

### CROSS REFERENCE TO RELATED APLICATIONS

This application is related to application Ser. No. 10/119,554 filed Apr. 10, 2002.

### BACKGROUND, FIELD OF THE INVENTION

The invention is a surgical instrument that provides a new and improved method for sternal closure. The new method is easier, more precise and safer than that as practiced in prior art.

### BACKGROUND, DISCUSSION OF PRIOR ART

Presently, sternal closure with surgical stainless steel sutures is practiced almost universally. The process is described in detail to allow comparison with the new method and apparatus. Sutures are supplied commercially with a large curved needle attached to a single end. The needle is grasped with an instrument called a needle driver. The surgeon is required to pass the needle through the sternum by applying a force to the needle driver with one hand, while applying an opposing force on the undersurface of the sternum with the other hand. Thus, the needle passes through the sternum in a direction, from outside grasped with the needle driver and pulled through to the inside. The suture needle must now be passed in a direction from inside to outside on the opposing side of the sternum, using the needle driver. Multiple sutures are placed in this manner to complete the closure.

Problems associated with this method of closure are listed below.
1. Significant stress is placed on the hands of the surgeon.
2. Bent or broken needles occur as a result of difficulty in passing a curved needle through the bony sternum.
3. There is frequently difficulty controlling the path of the curved needle, resulting in imprecise suture placement.
4. The surgeon's hand is placed in harms way for possible needle puncture and exposure to serious blood born disease.

### OBJECTS AND ADVANTAGES OF NEW SUTURE APPARATUS AND METHOD

1. The apparatus uses straight rather than curved needles, allowing for precise and predictable placement of sutures.
2. The mechanical advantage provided by the apparatus allows the sutures to pass through the sternum with relative ease, significantly reducing stress on the surgeon's hands.
3. The incidence of bent or broken needles is reduced.
4. The apparatus provides the opposing force required at the undersurface of the sternum, thus protecting the surgeon from possible needle puncture of the hand and the risk of exposure to blood born disease.
5. elimination of sharp needle tips provides further protection from needle puncture of hands.
   Further objects and advantages of the invention will become apparent from a consideration of the drawings and the ensuing description

Having considered the art in terms of method, it will be helpful to examine hardware; the instruments, sutures and needles used in the suturing process. The basic tool for passing sutures through tissues is called a needle driver. It is essentially a streamlined pliers, capable of locking jaws to hold a needle. The tool has changed little over the years. On the other hand, needles and sutures and their relationship have changed considerably. Needles and sutures evolved as separate entities. Needles were made with an eyelet. A strand of suture “threaded” the needle in preparation for use. Needles were sterilized and reused many times over.

By the early nineteen sixties technology had developed whereby a disposable needle was “swedged on” to its suture. Advantages included very sharp needles and streamlined passage through tissues. By the late nineteen sixties, all sutures were of this type. The sutures currently in use for sternal closure use such needles,

Sternal closure presents special problems. To begin, the needle must be passed through bone. In addition, there are serious space constraints as the heart lies immediately beneath the sternum. The ongoing use of curved needles relates to the space problem.

The advantages of the use of straight needles for sternal closure have been recited. The new apparatus and method permit such use by utilization of suture and straight needle as separate entities.

### SUMMARY OF THE INVENTION

The problems discussed above are addressed and at least partially solved with the improved suturing apparatus and methods. In a particular apparatus embodiment, disclosed is a handled framework that supports opposing, coactive male and female engagement elements. The female engagement element carries a suture and fits into a socket in which it is secured in a precise position. The male engagement element is a straight surgical needle carried in an arm and reciprocated to the handled framework in opposition to the female engagement element. A drive assembly attached to the handled framework is capable of imparting linear motion to the arm. Preferably, the drive assembly includes a set of inclined teeth or detents, carried by the arm, a pawl capable of interacting with the inclined teeth and a lever, pivoted to the handled framework and thence to the pawl. The pawl is moveable in reciprocal directions in response to pivotal movement of the lever. Lever motion imparts linear motion to the arm and the needle carried therein.

A needle guide or stabilizing device is moveably attached to a perpendicular extension of the handled framework. The needle guide is positioned intermediate to the male and female engagement elements. The guide carries an aperture through which the surgical needle must pass prior to penetration of the sternum, en route to the female engagement element. The method for using the suture apparatus herein disclosed will now be summarized.
The apparatus is positioned to place the sternum intermediate to, the male and female engagement elements.
A force is applied to the lever and repeated as necessary to accomplish penetration of the sternum and engagement of the male and female engagement elements. The lever will bias to the open position as manual pressure is released.
As linear movement is imparted to the arm and the needle it carries, the needle passes sequentially through the needle guide and bony sternum and then into the female engagement element. Although the needle tip is of slightly greater outside diameter than the inside diameter of the female engagement element, the needle penetrates and becomes trapped therein by friction The apparatus is then disengaged from the suture and removed from the site to be reloaded.
The needle can now be extracted in a retrograde direction using a conventional needle driver, thus delivering the suture through the sternum.
The steps described above are then repeated on the opposing side of the sternum, which completes the placement of a single suture.

### BRIEF DESCRIPTION OF THE DRAWINGS

FIG. 1 is a perspective view of the apparatus in its environment, between cut sternal edges.

FIG. 2 is a side view of the apparatus showing the engagement elements in position for use and hidden lines revealing portions of the drive assembly.

FIG. 3 is a side view of the apparatus showing the male engagement element having penetrated the sternum and the lever in closed position.

FIG. 4 is a frontal view of the apparatus, with the engagement elements in position for use.

FIG. 4A is a sectional view through section lines A-A Needle guide has not been moved to a lower position for use.

FIG. 5 is an exploded view of the needle guide as it mounts the perpendicular extension.

FIG. 5A is a sectional view through section lines D-D.

FIG. 6 shows the upper and forward portion of the handled framework and arm. Emphasis is directed to the exploded view of the friction device and its relation to the arm.

FIG. 6A is a sectional view through section lines C-C.

FIG. 7 is a perspective view of the fixture, as viewed from its inferior surface. Line D shows the position of the female engagement element seated in the socket. (Socket not shown). The circular arrowed lines refer to reciprocal rotary motion of the knob, which opens or closes the fixture.

FIG. 7A is a sectional view through section lines A-A.

FIG. 7B is a sectional view through section lines B-B.

FIG. 8 is an exploded view of the proximal, bifurcate portion of the lever, also showing the pawl and pivot pin that fit into the space created by the bifurcation.

FIG. 8A shows perspective views of one embodiment of the male and female engagement elements.

FIG. 9 is a perspective view of a second preferred embodiment of the needle guide showing the grooved piston in its cylinder and male engagement element in place.

FIG. 9A is a sectional view through section lines E-E.

### DETAILED DESCRIPTION OF THE INVENTION

The preferred embodiment includes: the handled framework 251, a friction device 274, a needle guide 255, the arm 270, the drive assembly 280, a fixture 253, male engagement element 252, and female engagement element 254. In order to make the more detailed description, which follows as simple, clear and concise as possible, the components will be described separately. It should be noted, that in describing one component, it is often necessary to make reference to another.

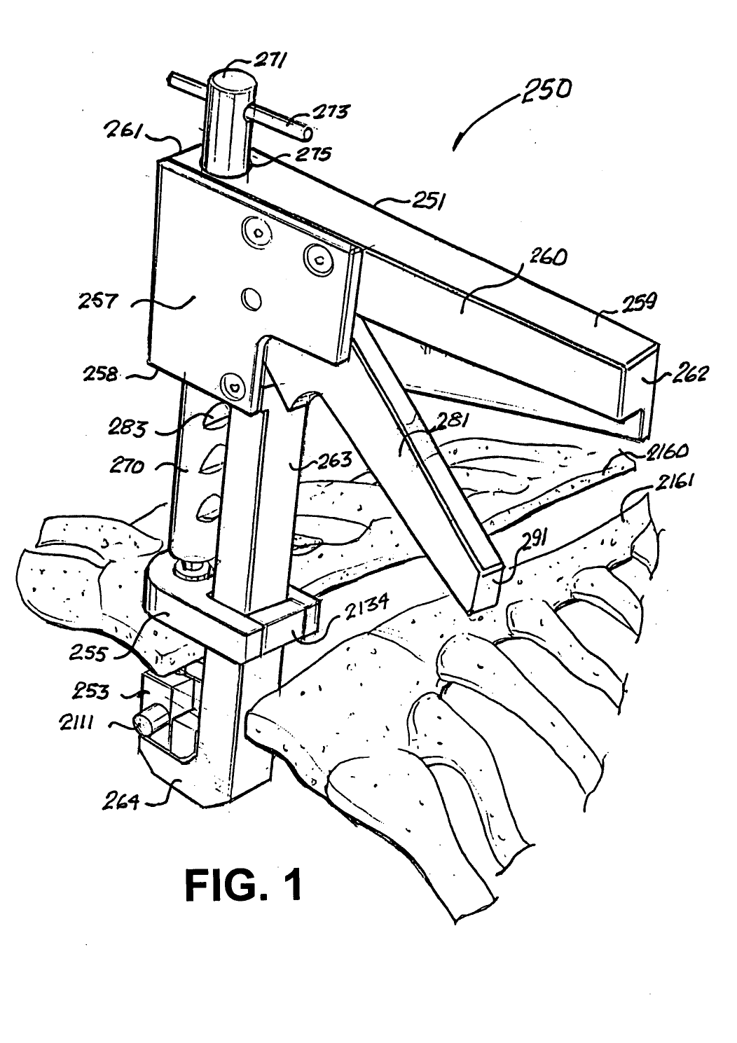

As shown in FIG. 1, handled framework 251 includes a handle 260 with opposing forward extremity 261 and a rearward extremity 262. A perpendicular Extension 263 is offset rearward of forward extremity 261 and extends distally, terminating at distal extremity 264. An enlarged seat 265 extends forward at distal extremity 264. Forwardly and inferiorly, a horizontal margin 258 of handle 260 extends anteriorly from leading edge 266 to forward extremity 261 and defines the lowermost part of handle 260.

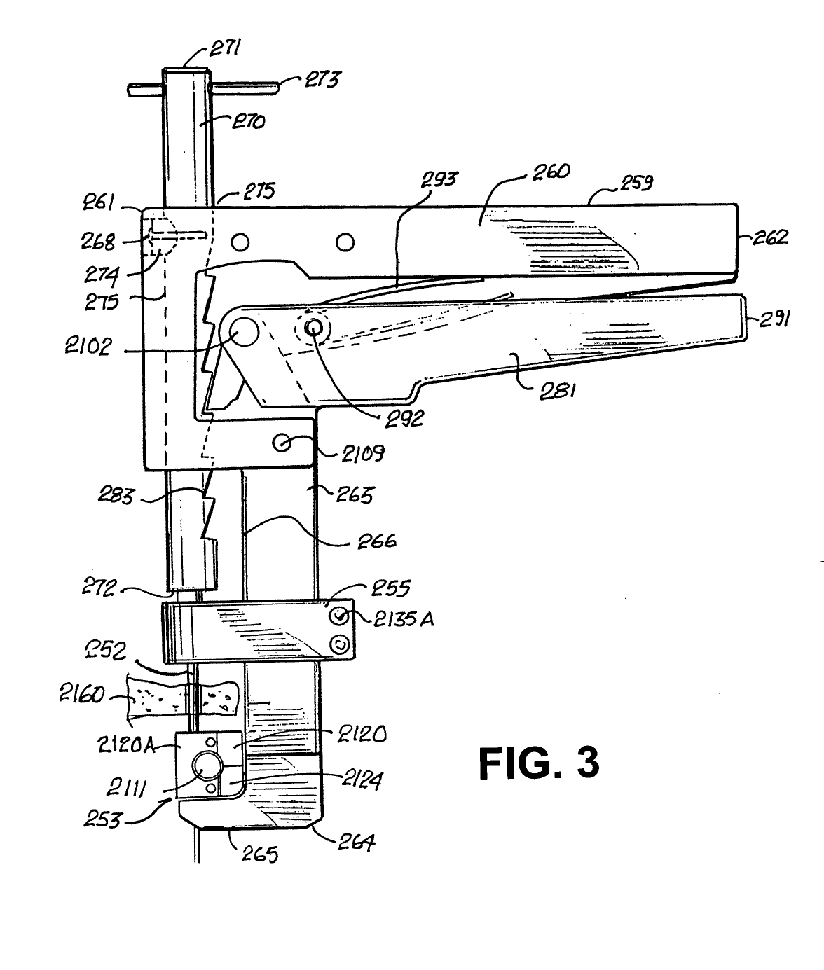

As best seen in FIG. 3, framework 251 supports a channel 275, which passes therethrough and is perpendicular to handle 260 as illustrated. Arm 270 is disposed within and extends through channel 275 and is capable of reciprocal movement therein, in opposition to female engagement element 253.

Framework 251 further supports a friction device 274 located at forward extremity 261, inferior to top surface 259. The friction device is cylindrical and carries a flat proximal end 279, and a concave distal end 278. The proximal end carries medial and lateral shoulders, 279 A and B. A pair of threaded bores, 267A and B extends horizontally into framework 251 commencing at a forward extremity 261 of the handled framework. The bores are positioned at the medial and lateral margins of friction device 274, which permits the heads of screws 268A and 268B to overlap shoulders 279A and 279B. It can be seen that by adjustments of the screws, the friction device 278 can be moved relative to arm 270. Such movement governs the degree of friction between the device and the arm. The friction device is not considered essential to successful function of the tool.

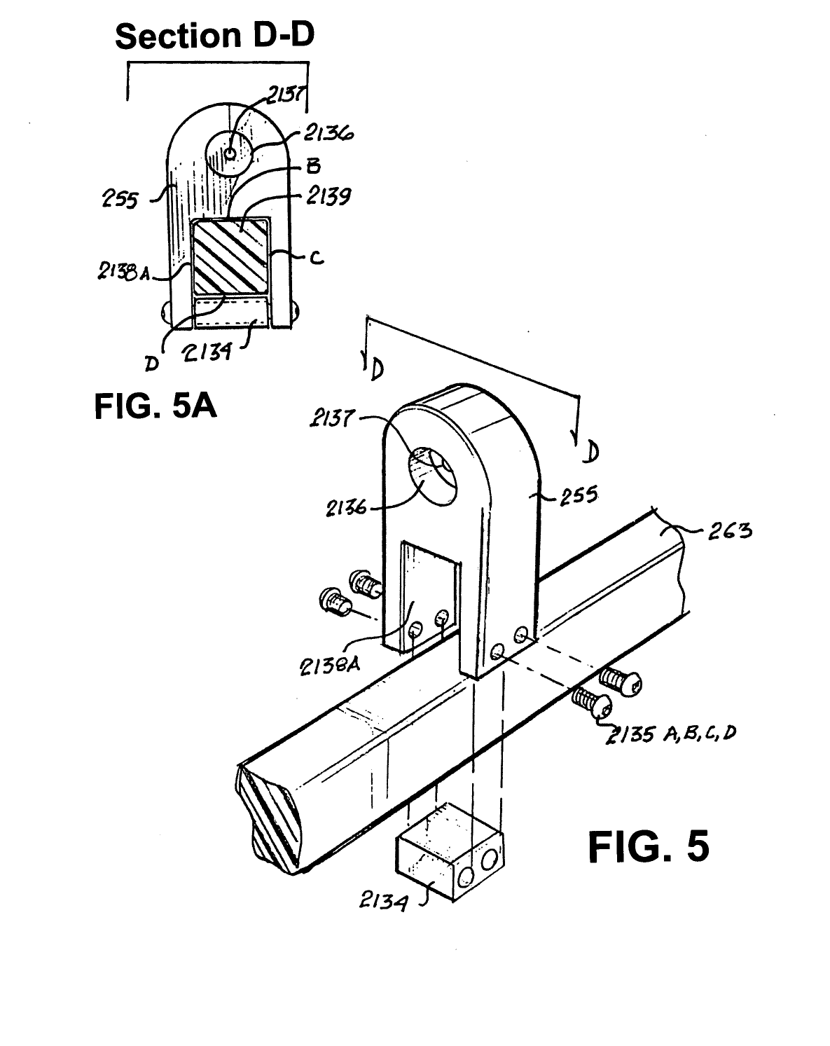

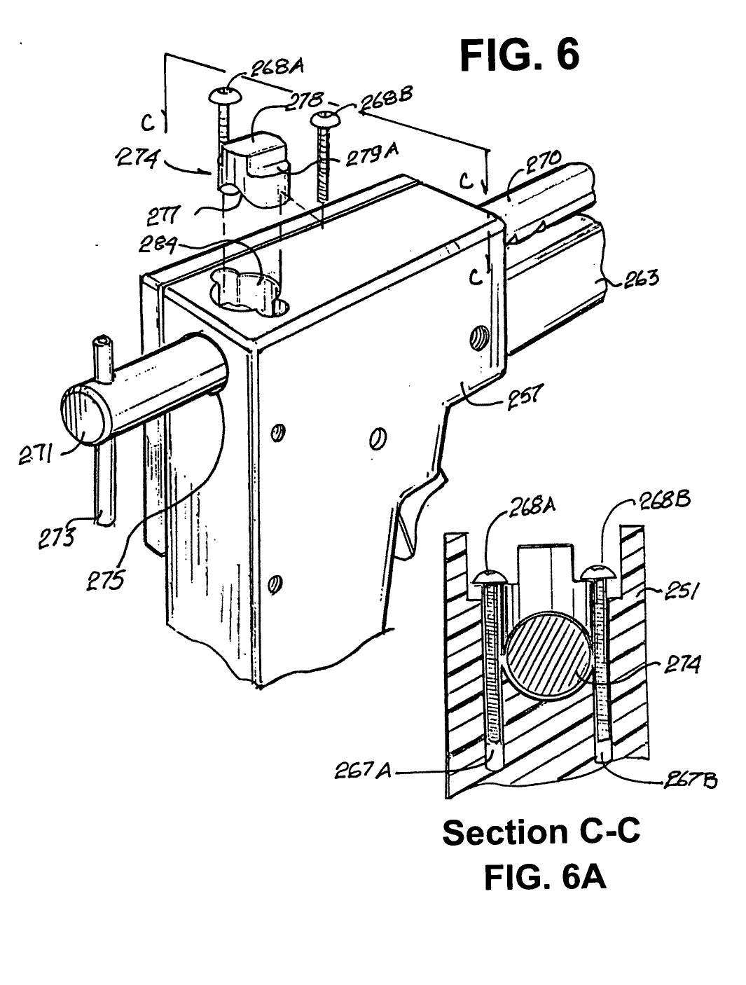

Handled framework 251 further carries a needle guide 255, moveably mounted to the perpendicular extension 263. The device functions to support and precisely guide a surgical needle 252, to its target, a female engagement element, 254. The needle guide is horseshoe shaped, with its outer aspect defining a convex curve anteriorly, while limbs extend posteriorly like a horseshoe. A Bridge 2134, connects the posterior limbs using four screws 135A, B, C and D. With the bridge in place, four walls define an interior rectangular space, 2139. Refer to FIGS. 5and 5A. The outer surfaces of perpendicular extension 263 oppose the inner surfaces, 2139A,B,C and D of space 2139, allowing the needle guide to slide relative to the perpendicular extension. A manual force is required to impart movement to the needle guide.

At its anterior midline, the device is penetrated by two concentric holes which align with needle 252. Superiorly a larger hole, 2136 penetrates the majority of the way through the device and fits arm 70. Inferiorly a concentric smaller hole, 2137 completes the passage and fits needle 252 at its larger shank 2150.

Framework 251 further supports a space 285 bounded anteriorly by arm 270, superiorly by handle 260, inferiorly by uppermost part of extension 263 and laterally on one side by a continuous extension of handle 260. On the opposing lateral side, a flat, removable plate, 257 is attached by screws, 2109A,B and C. Posteriorly the space is open to accommodate lever 281. The space houses the following parts: lever 281 with a pivot pin 292 and a spring 293, pawl 282 with a pivot pin 2102 and a spring 2105 and arm 270 with inclined teeth 283.

Handled framework may be constructed of various materials, including so called engineered plastics, capable of withstanding autoclave temperatures and meeting FDA approval. Plastics may be molded or machined. Metals of choice include surgical stainless steel, titanium or other appropriate alloys. Of course, a combination of materials may be used.

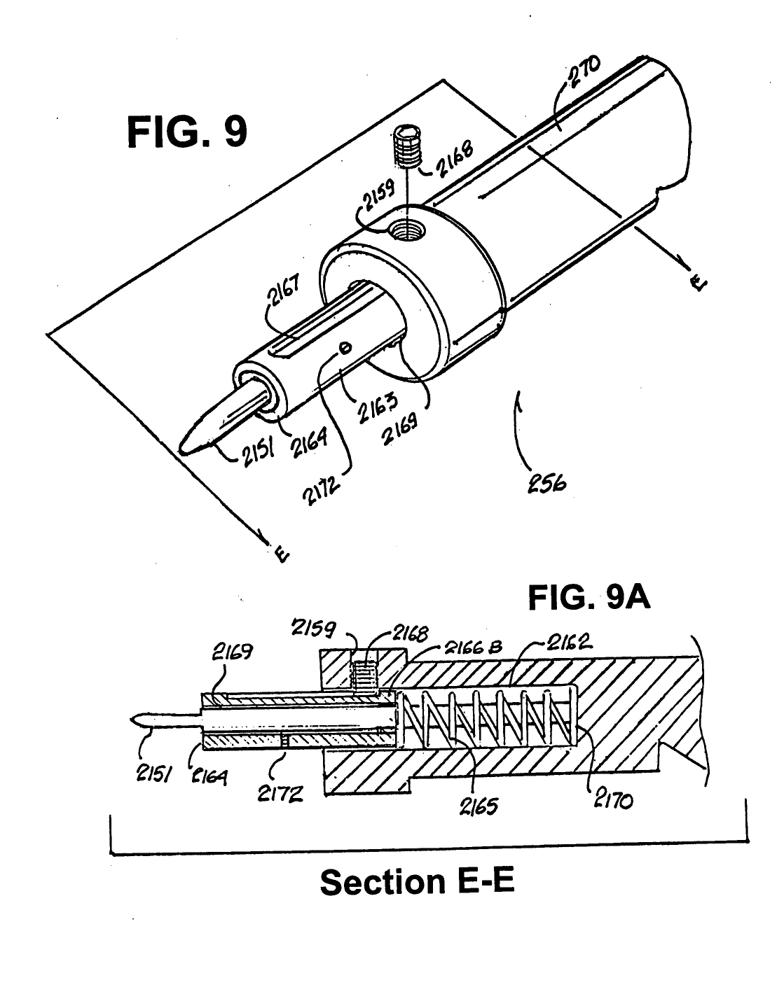

A second preferred embodiment of needle guide 255 is presented and labeled 256. Please refer to FIGS. 9 and 9A. The device utilizes a piston and cylinder arrangement, wherein a cylinder 2162, is created in distal end of arm 270, parallel to the long axis of arm 270. The cylinder 2162, carries a piston 2163. Piston 2163 carries a needle bore, 2169 passing through its long axis. A compression spring 2165, is positioned between proximal end of piston 2163 and blind end 2170, of cylinder 2162. Piston 2163 carries a groove 2167, on its outer surface. Groove 2167 parallels the long axis of piston 2163, and it terminates short of the proximal and distal ends of the piston, thus creating abutments 2166A and 2166B. Distal arm 270 carries a threaded bore 2159, supporting a set screw 2168, which intercepts channel 2167. With set screw 2168 positioned in groove 2167, piston 2163 can move along groove 2167, but its most distal position is defined by the contact of set screw 2168 and abutment 2166B. Compression spring 2165 functions to keep piston 2163 in this most distal position at rest.

A functional description follows. A straight surgical needle 252, is inserted into needle bore 2169. The proximal end 2152 of the needle abuts the blind end 2170 of cylinder 2162. The apparatus is positioned in relation to the sternum. A force in applied to lever 281 as needed, thus moving the needled end 2153 of needle 252 to penetrate the sternum 2160. Upon further penetration, the collar 2164, of piston 2163, contacts the upper surface of the sternum exerting a force on piston 2163, forcing it ever deeper into cylinder 2162. All the while however, as needle 252 continues its penetration, it is being supported by piston 2163, the collar 2164, of which remains in contact with the sternum.

The arm is an elongate rigid device with opposing proximal end 271 and distal end 272. Proximal end 271 carries a handle, 273. Handle 273 may be used to impart two separate types of motion to arm 270. By twisting the handle, a rotary motion is imparted to arm 270, which serves to disengage the inclined teeth 283, from pawl 282. By pushing or pulling on the handle, up or down, linear motion is imparted, allowing arm 270 to be positioned as needed.

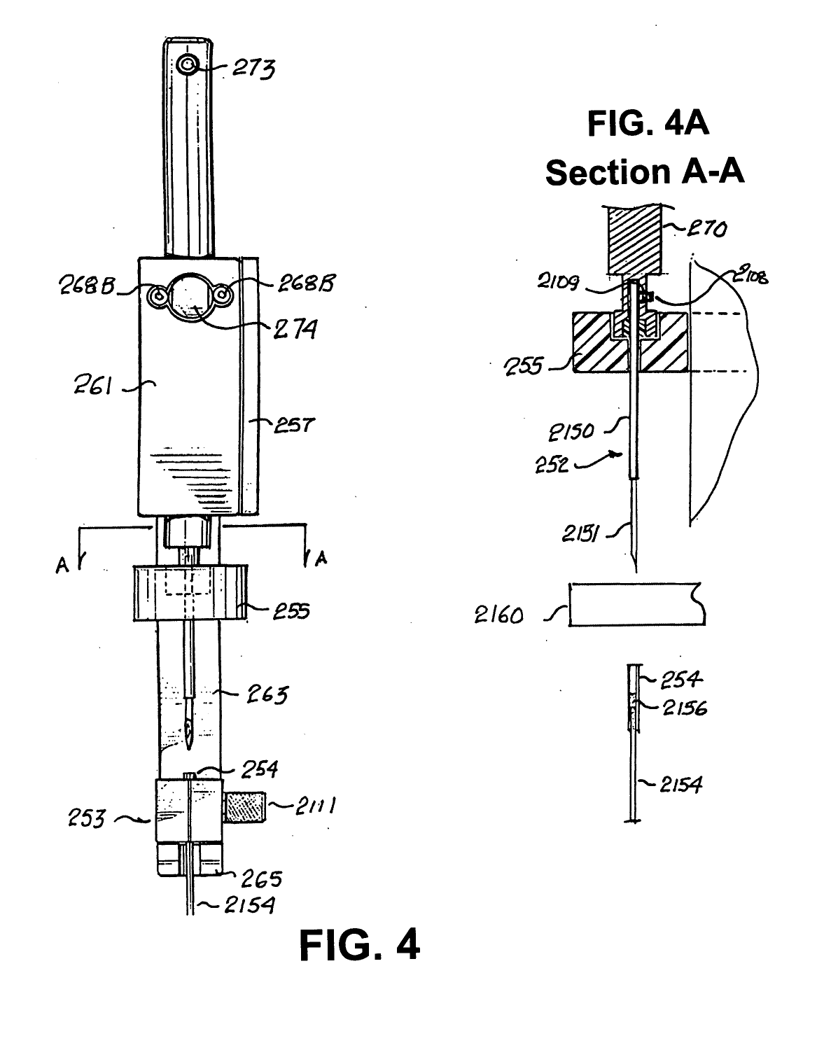

A second preferred embodiment for arm 270 is a mechanism for securing needle 252 in blind bore 276. A set screw 2108, passing through a threaded bore 2109 intercepts needle 252, locking the needle in position. Please refer to FIG. 4A. After male engagement element 252, and female engagement element 253 become secured, handle 273 can be used to extract suture 252 from the sternum.

The foregoing modification to arm 270 is applicable only when the apparatus utilizes the preferred embodiment of the needle guide 255.

However, the same end may be accomplished when the apparatus utilizes alternative embodiment 256 of needle guide. In this case, a set screw 2172 intercepts channel 2169 and needle 252 to accomplish the same goal.

Framework 251 carries an attached drive assembly 280 ( refer to FIGS. 1,2 and 3) that is capable of imparting motion to arm 270, thus moving a male engagement element, 252 toward a female engagement element 254, resulting in an interaction which will be described in detail later. Drive assembly 280 is a rachet arrangement which includes a lever, 281 pivoted to framework 251, a pawl, 282 pivoted to lever 281 and inclined teeth, 283 carried by arm 270. Lever 281 is pivoted to framework 251, underlies handle 260 and is substantially coextensive with handle 260. Referring to FIG. 3, lever 281 is elongate and has a proximal end, 290 disposed toward forward extremity 261 and a distal end, 291 disposed toward rearward extremity 262. Proximal end, 290 extends in to framework 251, more specifically into a chamber, 286 of framework 251 that communicates with channel 275 as illustrated.

Lever 281 is pivoted between its proximal and distal ends, 290 and 291 as illustrated, by a pin, 292 attached to framework 251. The pivot point of lever 281 is close to its proximal end, 290. A spring, 293 encircles pin 292. The spring has opposing free ends, 293A and 293B. These interact with opposing portions of handle 260 and lever 281, thus biasing distal end, 291 of lever 281 away from rearward extremity, 262 of handle 260. At the same time, end 290 of lever 281 and pawl 282 are biased toward handle 260 and away from distal extremity 264. Those having regard for the art will readily appreciate that other spring forms or biasing arrangements can be used for introducing the described bias to lever 281.

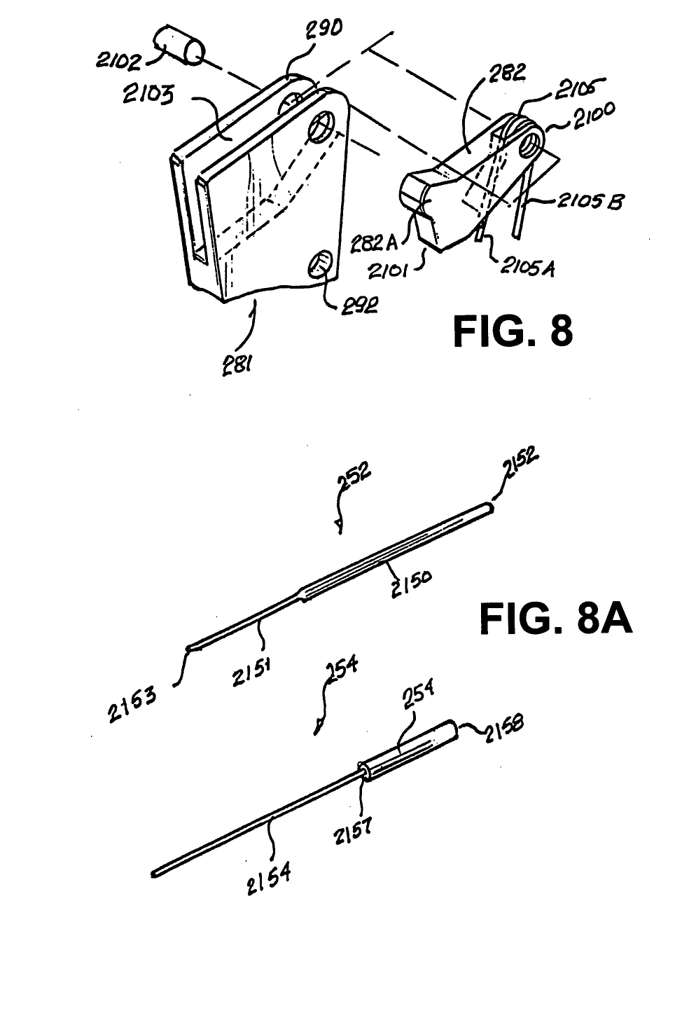

As previously mentioned, pawl 282 is pivoted to proximal end, 290 of lever 281. Arm 270 carries inclined teeth, 283. The teeth are disposed at spaced intervals along one side of the arm intermediate to ends 271 and 272. With reference to FIG. 8, pawl 282 has opposing ends, 2100 and 2101 plus a tongue, 282A disposed proximal to end 2101. End 2100 is pivoted to a pin, 2102 attached to proximal end, 290.of lever 281. Pawl 282 resides partially in a bifurcate feature, 2103 of lever 281, which characterizes proximal end, 290. Tongue 282A confronts and interacts with teeth 283. A spring, 2105 encircles pin 2102 and has opposing free ends, 105A and B. These interact with confronting portions of pawl 282 and lever 281, biasing pawl 282 toward teeth 283 and causing tongue, 282A to interact with teeth, 283. Spring 2105 maintains pawl 282 in a biased state against arm 270. Those having regard for the art will readily appreciate that other spring forms or biasing arrangements can be used for introducing the described bias to pawl 282.

Handle 260 and lever 281 are capable of being taken up by hand. By applying a manual force to lever 281, the bias of spring 293 is overcome and lever 281 is pivoted. This causes pawl 282 to reciprocate and interact with teeth 283 which forcibly moves arm 270 and engagement element 252 toward engagement element 253. When the manual force on lever 281 is released, spring 293 pivots lever 281 to an open position. That is to say, distal end 291 of the lever moves away from distal end 262 of the handle 260. At the same time pawl 282 moves upward (away from distal extremity 264) and out of engagement with a single tooth 283, and into engagement with a second single tooth located higher on arm 270. The second tooth is higher than the first on arm 270. By repeatedly squeezing and releasing the lever: handle combination, the pawl is repeatedly engaged and disengaged. Arm 270, therefore is moved downward in channel 275 (toward distal extremity 264). This, of course moves the engagement elements, 252 and 254 closer and closer together. Ordinarily, two or three strokes will engage the elements. Construction materials and methods are unchanged from those previously suggested.

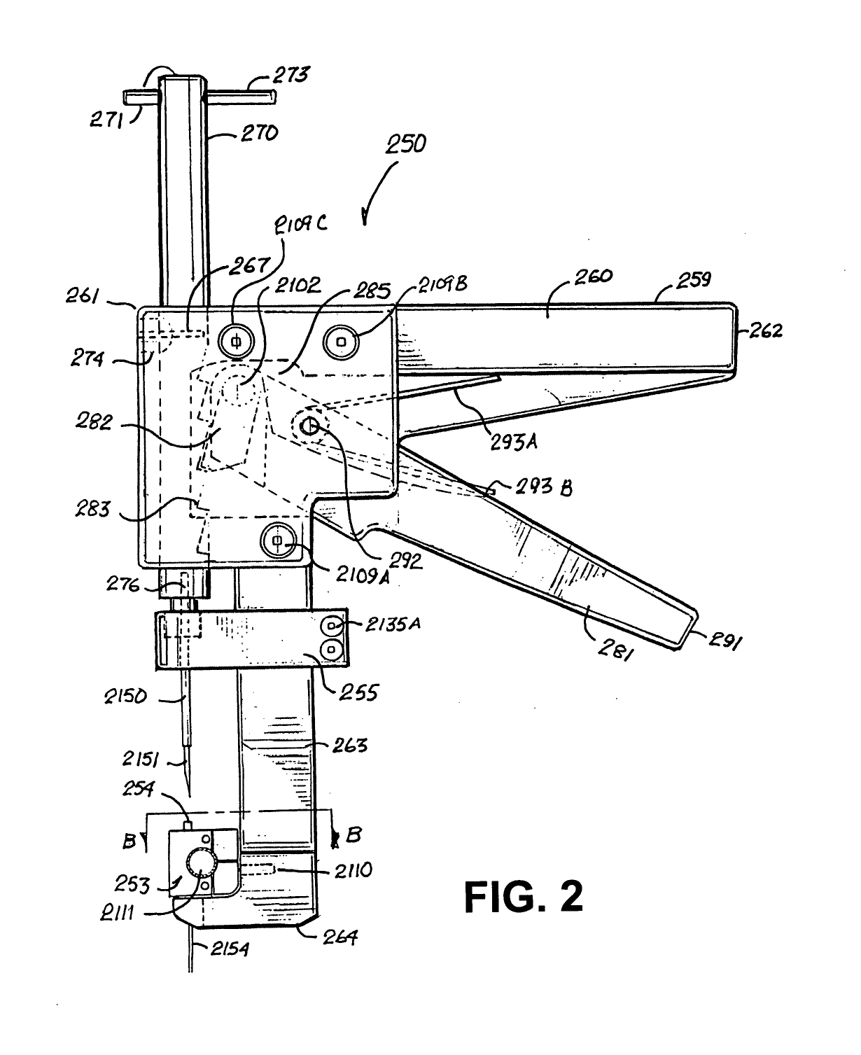

Details of the male engagement element 252 and the female engagement element 254 will now be considered. Refer to FIGS. 2, 3 and 8. Engagement element 252 is a surgical needle carried by arm 270 in blind bore 276. Alternately, when second preferred embodiment 256 is employed, needle 252 is then carried in bore 2169 in piston 2163. Needle 252 has a proximal, butt end 2152 and a distal penetrating end 2153. The needle has a longer and thicker proximal shank, 2150 and a shorter and thinner distal shank, 2151. This arrangement discourages needle failure by buckling or compression, while the size of the distal needle and female engagement element may be minimized. Penetrating end 2153 of the needle is elliptical rather than sharp. The female engagement element, 254 is a cylinder attached to a stainless steel surgical suture, 2154. Attachment of the suture to the cylinder may be accomplished with surgical adhesives, crimping or alternate methods. Inside diameter of the cylinder must be sized to accommodate distal end 2151 of needle 252 to extremely close tolerances.

fixture 253 is a housing to precisely position and secure the female engagement element 254 in a socket 2113. Socket 2113 has an upper, proximal end 2118, and a lower, distal end 2119. The socket is a cylindrical passage. It narrows abruptly at its distal end, creating a small gap 2104. The gap allows a suture 2154, which is attached to the female engagement element to pass there through, but blocks the female engagement element. The narrowed site provides a seat for the female engagement element.

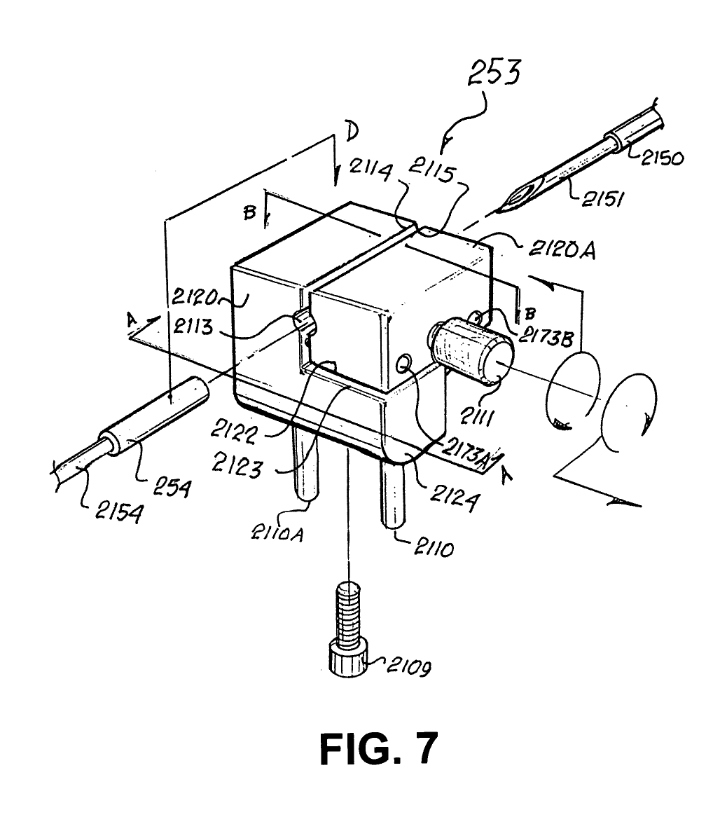

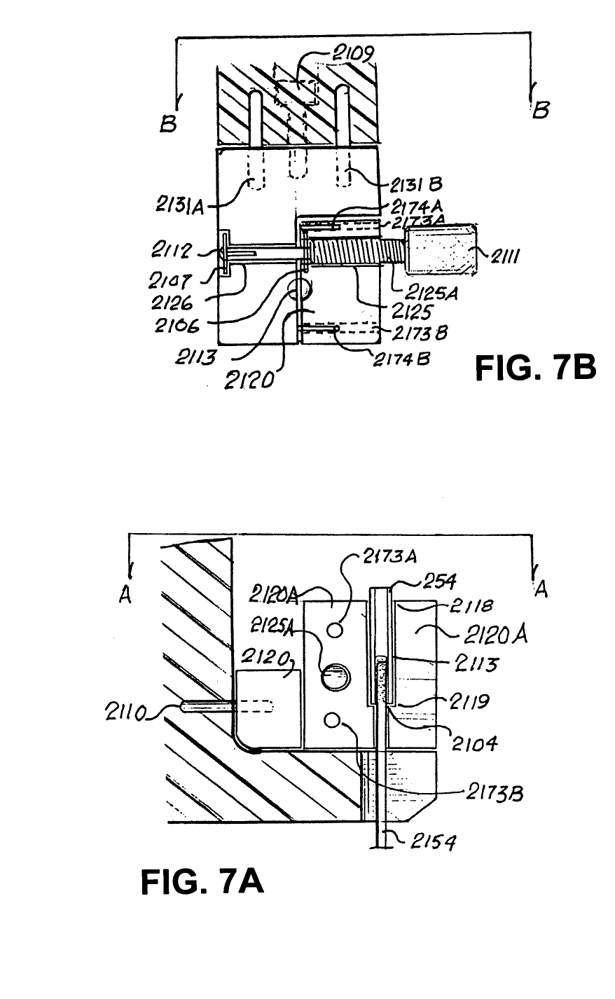

Fixture 253 includes two separate pieces. Please refer to FIGS. 7, 7A and 7B. A larger “L” shaped piece 2120 and a smaller rectangular piece 2120 A. The pieces, when joined together, form a rectangular block. The pieces interact and join at two interfaces. An opposing face 2114 of piece 2120 interfaces with opposing face 2115 of piece 2120A. This forms a plane of opposition oriented in a fore and aft direction. The plane exactly bisects socket 2113. A second interface is oriented at right angles to the first. The second interface is formed by opposing face 2122 of piece 2120A and opposing face 2123 of lateral extension, 2124 of piece 2120. Piece 2120 is fixed in position by two parallel pins 2110 and 2110A and screw 2109. Piece 2120 is thus secured to seat 265 of the handled framework 251. Piece 2120A is capable of medial and lateral (side to side) movement. Such movement permits socket 2113 to assume a wider disposition, assuring easy disengagement of the suture and female engagement element from the apparatus.

The mechanism by which such movement is generated will now be described. Movement of piece 2120A toward or away from piece 2120 occurs along a pair of guide pins, 2174A and B. A pair of bores 2173A and B traverse piece 2120A and the penetrate a short distance into piece 2120. The guide pins are stationarily secured in the bores in piece 2120. The guide pins slideably fit into the bores in piece 2120A. This arrangement allows movement of piece 2120A relative to piece 2120, which is fixed to the handled framework. A channel, 2125 traverses both piece 2120A and piece 2120. Its proximal portion, traversing piece 2120A is threaded, while its distal portion, traversing piece 2120 is not. A shaft 2125A traverses both portions of the channel and is threaded in its proximal portion to coact with the threads of the proximal channel. The shaft carries a knob 2111 at its threaded end to impart rotation to the shaft.

When a manual force is used to impart a rotary motion to knob 2111, shaft 2125A is rotated and a linear motion is created which moves piece 2120A toward or away from piece 2120, thus opening or closing socket 2113. Opening the socket permits the apparatus to be easily disengaged from the suture. Those familiar with the art will recognize other methods are available to accomplish the ends described, such as springs, hinges or manual manipulation to name a few.

The close tolerances required for proper finction of fixture 253 seem to make the metal alloys the materials of choice and machining the preferred method for construction. Molding of appropriate plastics might be possible.

Needles are produced using surgical stainless steel rods of appropriate diameter. The needle tips require precise sizing, therefore some machining is required. This can be accomplished using CNC mini lathes. Grinding is also an alternative. Forging methods with supplemental machining is also possible.

A word regarding the method for joining the suture to the female engagement element is necessary. Crimping is possible, but may cause deformity to the engagement element, which may interfere with proper fit in the socket. Epoxy resins work well, but if they are used, must be medical adhesives, biocompatible, USP class 6. A heat curable epoxy, Permabond 4E96 meets these criteria and is available from Permabond of Bridgewater, N.J.

1. An apparatus for passing sutures through a surgically divided sternum, comprising:
a handled framework supporting opposing male and female engagement elements; and
an arm which carries said male engagement element; and
a drive assembly and means to impart motion to the male engagement element to thereby penetrate a segment of the divided sternum; and
said handled framework further supporting a perpendicular extension and a seat and means to position a socketed fixture at the undersurface of the divided sternum, whereby said socket or contents therein may be penetrated by the male engagement element; and
the socket of said socketed fixture with means to seat and precisely position said female engagement element with said suture attached thereto.

2. The apparatus of claim one wherein the male engagement element is a straight surgical needle.

3. The apparatus of claim one wherein the female engagement element is preferably a cylindrical device, securely attached to opposing ends of each suture.

4. The female engagement element of claim three being of appropriate size to allow penetration by said straight surgical needle and assure frictional capture of needle therein.

5. Said arm of claim one further carrying a needle guiding device and means to support and guide the straight surgical needle to its target, the female engagement element.

6. Said needle guiding device of claim five, comprising a piston operationally joined to a cylinder in the distal portion of the arm, wherein said piston supports a bore to carry a straight surgical needle, thereby imparting stability and directional control to the needle.

7. The apparatus of claim one, wherein said handled framework comprises a handle member having forward and rearward extremities and said perpendicular extension extending downward from said forward extremity and terminating as an enlarged seat for said socketed fixture.

8. A channel, passing through the handled framework in parallel disposition, but anterior and superior to said perpendicular extension, provides slidable and pivotable attachment means for the arm to the handled framework.

9. The apparatus of claim one, wherein the arm has a series of inclined teeth along one side to operatively engage a pawl, pivotably attached to a lever, which in turn is pivotably attached to the handled framework.

10. The arm of claim eight further supports a handle at its proximal extremity to manually impart slidable or pivotable motion thereto.

11. The lever of claim nine using a spring for biasing means, coacting between the lever and the handle member normally urging the lever to a fully open disposition.

12. The pawl of claim nine, with spring biasing means normally urging the pawl to pivot upward to coact with said inclined teeth, thereby providing means to impart motion to the arm when the lever is taken up by hand.

13. The apparatus of claim one, wherein the socketed fixture comprises a fixed and a slidable member, whereby a fore and aft plane of division between the members bisects the fixtures socket.

14. The members of claim thirteen are operatively joined by means of laterally and medially oriented guidepins and further joined by a threaded channel operatively connected to a knobbed, threaded shaft, providing means for said slidable member to move toward or away from said fixed member, thereby altering the diameter of the socket.

15. The fixed member of claim thirteen having fastening means to said enlarged seat comprising guidepins and bores or other obvious alternatives.

16. The socket of claim thirteen having an upper, proximal extremity and a lower, distal extremity; divided by a narrow gap near said lower distal extremity, thereby allowing passage of the suture while providing a seat for the female engagement element.

17. The straight surgical needle of claim two, absent a threading eyelet, comprising a separate entity from the suture.

18. With male and female engagement elements secured one to the other by friction, a portion of the straight surgical needle remains protruding from the top surface of the sternum, whereby it may be extracted in a retrograde direction by means of the arm or a conventional needle driver, thereby delivering the suture to a desired position at the superficial surface of the sternum.

19. The method of claim eight providing means to pass the opposing end of the suture through the opposing site of the divided sternum.
